(19) United States
(12) Patent Application Publication
(10) Pub. No.: US 2025/0152882 A1
(43) Pub. Date: May 15, 2025

(54) TRACHEOSTOMY DILATOR

(71) Applicant: TELEFLEX LIFE SCIENCES UNLIMITED COMPANY, Athlone (IE)

(72) Inventors: Adrian MAHON, Walshisland (IE); Joanne FAYNE, Athlone (IE); Cathal TIGHE, Athlone (IE); Gavin REYNOLDS, Cloone (IE); Katy RYAN, Monkstown (IE)

(21) Appl. No.: 18/922,236

(22) Filed: Oct. 21, 2024

Related U.S. Application Data
(63) Continuation of application No. 17/525,940, filed on Nov. 14, 2021, now Pat. No. 12,121,658.
Application No. 17/525,940 is a continuation of application No. PCT/IB2020/054638, filed on May 15, 2020.
(60) Provisional application No. 62/848,310, filed on May 15, 2019.

(51) Int. Cl. A61M 16/04

(52) U.S. Cl. CPC A61M 16/0472; A61M 16/0475

(57) ABSTRACT

A tracheostomy dilator may include a distal portion, a handle portion, a lumen, and a proximal portion. The distal portion may have a curve extending to a distal opening and be configured to dilate a tracheostomy. The handle portion may be proximal of the distal portion and be configured to be grasped by a user. The lumen may be configured to receive a guidewire. The proximal portion may be proximal of the handle portion and have a proximal opening in communication with the lumen. The proximal portion may be configured to deflect the guidewire at an angle of at least 30 degrees from a longitudinal axis of the handle portion.

### PRIORITY

The present application is a continuation of U.S. application Ser. No. 17/525,940, filed Nov. 14, 2021, which is based on International Application No. PCT/IB2020/054638, filed on May 15, 2020, which claims the benefit of priority to U.S. Provisional Patent Application No. 62/848,310, filed on May 15, 2019, and now expired, the disclosures of which are expressly incorporated herein by references in their entireties.

### TECHNICAL FIELD

The present disclosure relates generally to dilators, and more particularly to percutaneous tracheostomy dilators configured to reduce or prevent pinching of a guidewire.

### BACKGROUND

Percutaneous tracheostomies can be formed by inserting a hollow needle through the skin into the trachea and inserting a guidewire along the needle in the opening. The needle is then withdrawn, and a tracheostomy dilator is passed over the guidewire to expand the opening for the insertion of a tracheostomy tube. The present invention solves one or more problems involving the tracheostomy dilators of the prior art.

### OVERVIEW

One aspect of the present invention is directed to a tracheostomy dilator including a distal portion, a handle portion, a lumen, and a proximal portion. The distal portion may have a curve extending to a distal opening and be configured to dilate a tracheostomy. The handle portion may be proximal of the distal portion and be configured to be grasped by a user. The lumen may be configured to receive a guidewire. The proximal portion may be proximal of the handle portion and have a proximal opening in communication with the lumen. The proximal portion may be configured to deflect the guidewire at an angle of at least 30 degrees from a longitudinal axis of the handle portion.

In some embodiments, the angle may be at least 45 degrees from the longitudinal axis of the handle portion. In some embodiments, the handle portion may be substantially straight. In some embodiments, the handle portion may be textured. In some embodiments, the handle portion may include a textured sleeve. In some embodiments, the distal portion may have a wall thickness that decreases along a length of the distal portion. In some embodiments, the tracheostomy dilator may further include an indicator of a size of the tracheostomy dilator on the distal portion. In some embodiments, the proximal opening may be disposed at the angle from the longitudinal axis. In some embodiments, the proximal portion may have a bend extending from the longitudinal axis to the angle from the longitudinal axis. In some embodiments, the proximal portion may be longitudinally aligned with the longitudinal axis of the handle portion. In some embodiments, the proximal portion may have a bevel in a circumferential wall at the proximal opening, such that the circumferential wall extends further proximally at an upper portion than a lower portion. In some embodiments, the bevel may be curved. In some embodiments, the proximal portion may have a hole distal of the proximal opening and in communication with the proximal opening, and the hole is configured to receive the guidewire. In some embodiments, the proximal portion may have a slot connecting the proximal opening and the hole, and the slot may have a width less than the proximal opening and the hole. In some embodiments, the proximal portion may be rotatable around the longitudinal axis relative to the handle portion. In some embodiments, the proximal portion may be detachable from the handle portion. In some embodiments, the proximal portion may include an insert having a tubular segment received within the handle portion to detachably secure the proximal portion to the handle portion.

Another aspect of the present invention is directed to a tracheostomy dilator including a distal portion, a handle portion, a lumen, and a proximal portion. The distal portion may have a curve extending to a distal opening and be configured to dilate a tracheostomy. The handle portion may be proximal of the distal portion and be configured to be grasped by a user. The lumen may be configured to receive a guidewire. The proximal portion may be proximal of the handle portion and have a proximal opening in communication with the lumen. The proximal portion may have a bevel.

In some embodiments, the bevel may be in a circumferential wall at the proximal opening, such that the circumferential wall extends further proximally at an upper portion than a lower portion. In some embodiments, the proximal portion may be longitudinally aligned with the longitudinal axis of the handle portion. In some embodiments, the proximal portion further may include a hole distal of the proximal opening and in communication with the proximal opening, and the hole is configured to receive the guidewire. In some embodiments, the proximal portion may include a slot connecting the proximal opening and the hole, and the slot may have a width less than the proximal opening and the hole.

### BRIEF DESCRIPTION OF THE DRAWINGS

In order that the invention may be readily understood, aspects of this invention are illustrated by way of examples in the accompanying drawings.

FIGS. 1A-1B illustrate a percutaneous insertion of a tracheostomy dilator into a tracheostomy.

FIGS. 2A-2C illustrate a first exemplary embodiment of a tracheostomy dilator according to the present invention.

FIGS. 3A-3C illustrate a second exemplary embodiment of a tracheostomy dilator according to the present invention.

FIGS. 4A-4C illustrate a third exemplary embodiment of a tracheostomy dilator according to the present invention.

FIGS. 5A-5C illustrate a fourth exemplary embodiment of a tracheostomy dilator according to the present invention.

FIGS. 6A-6D illustrate a fifth exemplary embodiment of a tracheostomy dilator according to the present invention.

The same reference numbers may be used in the drawings and the following detailed description to refer to the same or similar parts.

### DETAILED DESCRIPTION

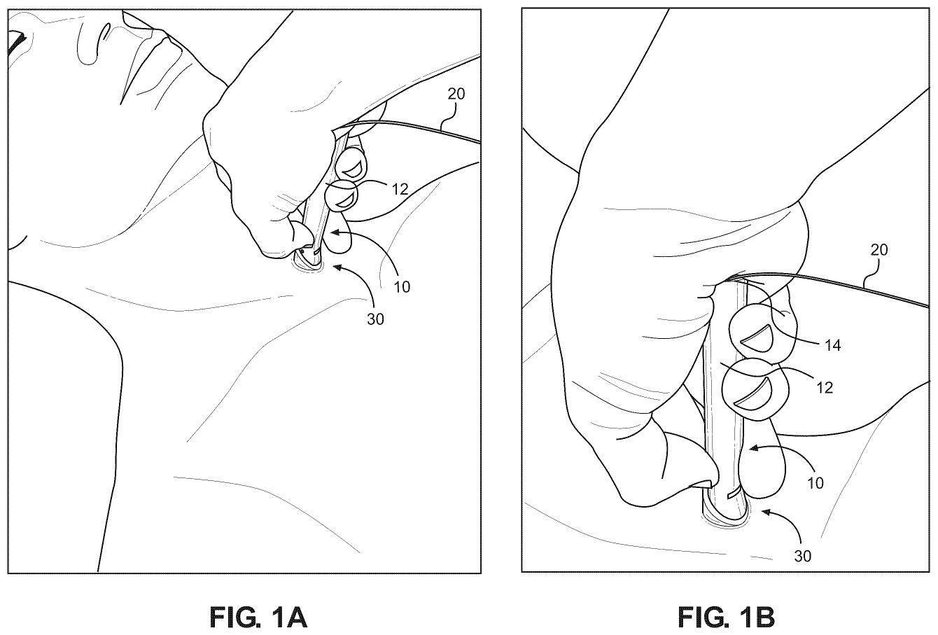

FIGS. 1A-1B illustrate a tracheostomy dilator 10 inserted over a guidewire 20 into a tracheostomy 30. A distal end (not shown) of the tracheostomy dilator 10 is configured to dilate the tracheostomy 30 for insertion of a tracheostomy tube (not shown). A handle portion 12 of the tracheostomy dilator 10 is grasped by a user as the tracheostomy dilator 10 is inserted over the guidewire 20 into the tracheostomy 30. As the tracheostomy dilator 10 is advanced over the guidewire 20, the guidewire 20 exits through a proximal opening 14 at the proximal end of the handle portion 12. The proximal opening 14 is disposed along the longitudinal axis of the handle portion 12, such that as the user pushes the tracheostomy dilator 10 into the tracheostomy 30, the user's palm tends to cover the proximal opening 14 and/or to pinch the guidewire 20 against the proximal end of the handle portion 12. This accidental pinching of the guidewire 20 may cause a number of complications including the undesirable pushing the guidewire 20 further into the trachea as the tracheostomy dilator 10 is advanced and/or pulling the guidewire 20 out of the trachea as the tracheostomy dilator 10 is removed. The present disclosure is directed to mitigating and/or overcoming one or more of these problems, and/or other problems associated with tracheostomy dilators and/or dilators in general.

The present disclosure is directed to tracheostomy dilators configured to shield and/or deflect the guidewire away from the longitudinal axis of the handle portion to reduce or prevent interference from the hand of the user. In some embodiments, the tracheostomy dilators may deflect the guidewire at least 30 degrees from the longitudinal axis of the handle portion, while the handle portion is substantially straight to facilitate grasping. In some embodiments, the tracheostomy dilators may deflect the guidewire at least 45 degrees from the longitudinal axis of the handle portion. The deflection of the guidewire may be caused by a bend at a proximal portion the tracheostomy dilator between the handle portion and a proximal opening. The bend may include a sharp bevel and/or a curve. In some embodiments, the proximal portion may be beveled at a face of the proximal opening, such that a circumferential wall of the proximal portion extends further proximally at an upper portion than a lower portion, effectively shielding the guidewire when grasped from above. In some embodiments, the proximal portion may, additionally or alternatively, include a hole through the bottom of the bottom portion and in communication with the proximal opening. The hole may connect to the proximal opening with a narrow slot, such that the guidewire may be received in the hole to reduce or prevent the chance of pinching when the user grasps the handle from above. In some embodiments, the proximal portion may be rotatable around the longitudinal axis of the handle portion and/or detachable from the handle portion. Thus, the various embodiments of the tracheostomy dilator may shield and/or deflect the guidewire to reduce or prevent pinching of the guidewire by the hand of the user against the proximal end of the tracheostomy dilator.

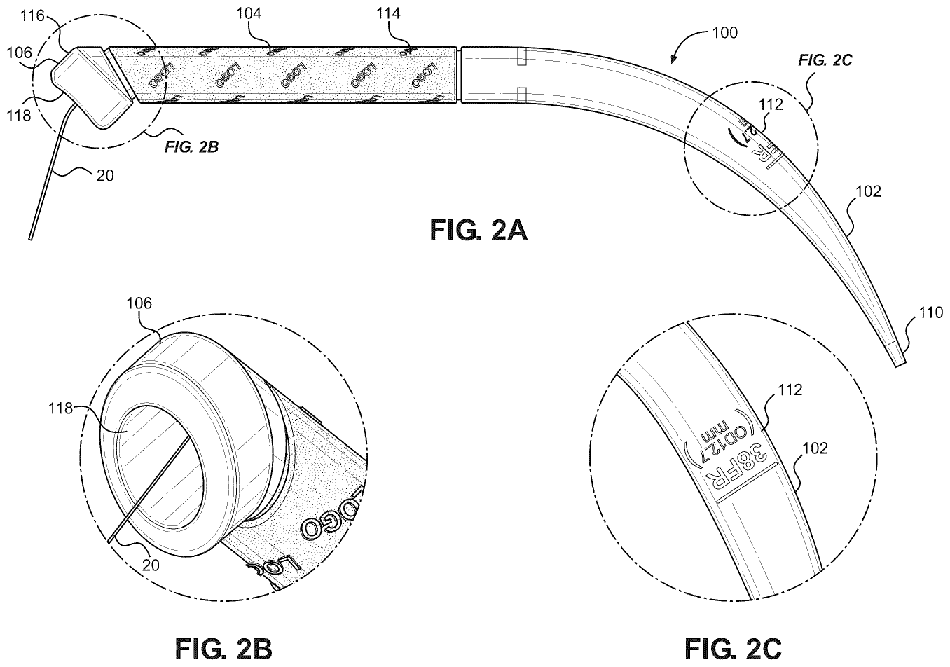

FIGS. 2A-2C illustrate a first exemplary embodiment of a tracheostomy dilator 100. The tracheostomy dilator 100 may include a distal portion 102, a handle portion 104, a proximal portion 106, and a lumen extending therethrough.

The distal portion 102 may including a continuous curve extending from the handle portion 104 to a distal tip 110 having a distal opening of the lumen. The continuous curve may enable the tracheostomy dilator 100 to pass through the trachea during insertion and for the distal tip 110 to clear the posterior tracheal wall. The distal portion 102 may taper along the continuous curve to the distal tip 110 to facilitate insertion. For example, the distal portion 102 may taper through a wall thickness that gradually reduces along the continuous curve to the distal tip 110. The distal portion 102 may have a hydrophilic coating to reduce friction during insertion into the tracheostomy, obviating the need for lubricating jelly. The distal portion 102 may also include indicia 112 of a size of a cross-section and a depth of the distal portion 102. For example, as illustrated in FIG. 2C, the indicia 112 may include a diameter (e.g., 38 French) and a line at least partially around the circumference indicating a proper depth for insertion of the distal portion 102 to dilate the tracheostomy.

The handle portion 104 may be substantially straight and include an elongated linear shaft integrally formed with the distal portion 102. The handle portion 104 may be proximal of the distal portion 102 and be configured to be grasped by a user. The handle portion 104 may be textured to increase grip and handling of the user. For example, the handle portion 104 may include a textured rubber sleeve 114 over the shaft. The grip on the textured surface may be enhanced by embossing a plurality of raised words and/or logos (e.g., of the manufacturer and/or distributor) along the length of the handle portion 104 and/or sleeve 114, The sleeve 114 may be received in an elongate recess on the outer surface of the shaft to prevent relative slipping.

The proximal portion 106 may be configured to deflect a guidewire 20 at an angle of least 30-45 degrees from a longitudinal axis of the handle portion 104. The proximal portion 106 may be integral to the elongated linear shaft of the handle portion 104 and/or the distal portion 102. The proximal portion 106 may include a bevel 116 that disposes a longitudinal axis of a proximal opening 118 of the proximal portion 106 at the angle of at least 30-45 degrees from the longitudinal axis of the handle portion 104. The deflected guidewire 20 may pass out of the proximal opening 118 of the tracheostomy dilator 100 when advanced. The proximal portion 106 may have a round cross-section with an outer surface and a lumen having a longitudinal axis disposed at the angle of least 30-45 degrees from a longitudinal axis of the handle portion 104. Due to the relative angle of the proximal portion 106 and proximal opening 118, pinching of the guidewire 20 by fingers and/or palms is reduced or prevented when the user grasps the handle portion 104.

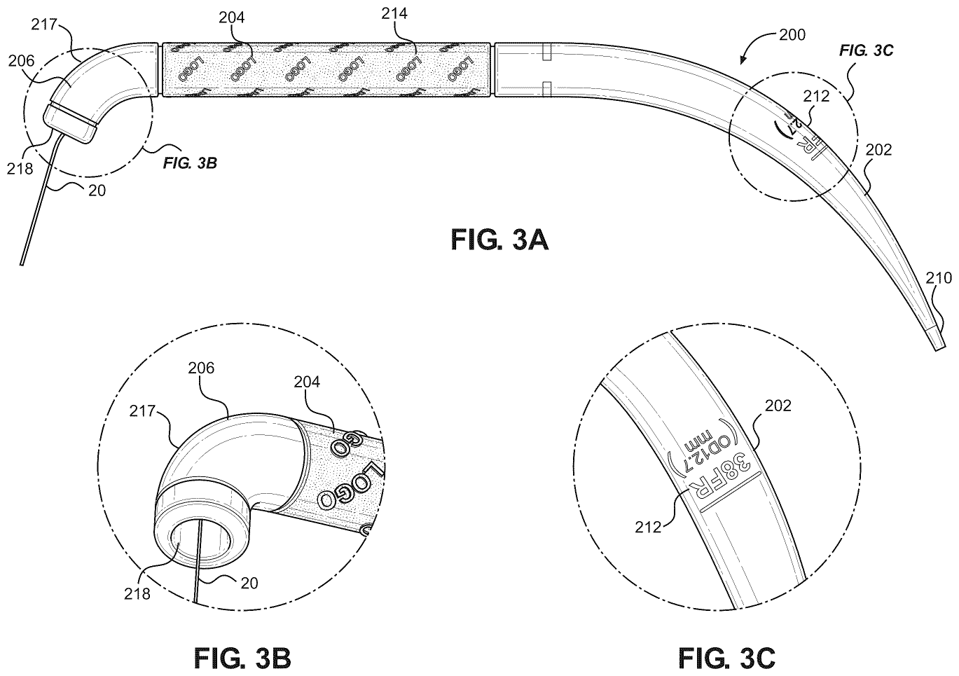

FIGS. 3A-3C illustrate a second exemplary embodiment of a tracheostomy dilator 200. The tracheostomy dilator 200 may include a distal portion 202, a handle portion 204, a proximal portion 206, and a lumen extending therethrough.

The distal portion 202 may including a continuous curve extending from the handle portion 204 to a distal tip 210 having a distal opening of the lumen. The continuous curve may enable the tracheostomy dilator 200 to pass through the trachea during insertion and for the distal tip 210 to clear the posterior tracheal wall. The distal portion 202 may taper along the continuous curve to the distal tip 210 to facilitate insertion. For example, the distal portion 202 may taper through a wall thickness that gradually reduces along the continuous curve to the distal tip 210. The distal portion 202 may have a hydrophilic coating to reduce friction during insertion into the tracheostomy, obviating the need for lubricating jelly. The distal portion 202 may also include indicia 212 of a size of a cross-section and depth of the distal portion 202. For example, as illustrated in FIG. 3C, the indicia 212 may include a diameter (e.g., 38 French) and a line at least partially around the circumference indicating a proper depth for insertion of the distal portion 202 to dilate the tracheostomy.

The handle portion 204 may be substantially straight and include an elongated linear shaft integrally formed with the distal portion 202. The handle portion 204 may be proximal of the distal portion 202 and be configured to be grasped by a user. The handle portion 204 may be textured to increase grip and handling of the user. For example, the handle portion 204 may include a textured rubber sleeve 214 over the shaft. The grip on the textured surface may be enhanced by embossing a plurality of raised words and/or logos (e.g., of the manufacturer and/or distributor) along the length of the handle portion 204 and/or sleeve 214, The sleeve 214 may be received in an elongate recess on the outer surface of the shaft to prevent relative slipping.

The proximal portion 206 may be configured to deflect a guidewire 20 at an angle of least 30-45 degrees from a longitudinal axis of the handle portion 204. The proximal portion 206 may be integral to the elongated linear shaft of the handle portion 204 and/or the distal portion 202. The proximal portion 206 may include a curve 217 that disposes a longitudinal axis of a proximal opening 218 at the angle of at least 30-45 degrees from the longitudinal axis of the handle portion 204. The deflected guidewire 20 may passes out of the proximal opening 218 of the tracheostomy dilator 200 when advanced. The proximal portion 206 may have a round cross-section with an outer surface curving to the angle of least 30-45 degrees from a longitudinal axis of the handle portion 204. Due to the relative angle of the proximal portion 206 and proximal opening 218, pinching of the guidewire 20 by fingers and/or palms is reduced or prevented when the user grasps the handle portion 204.

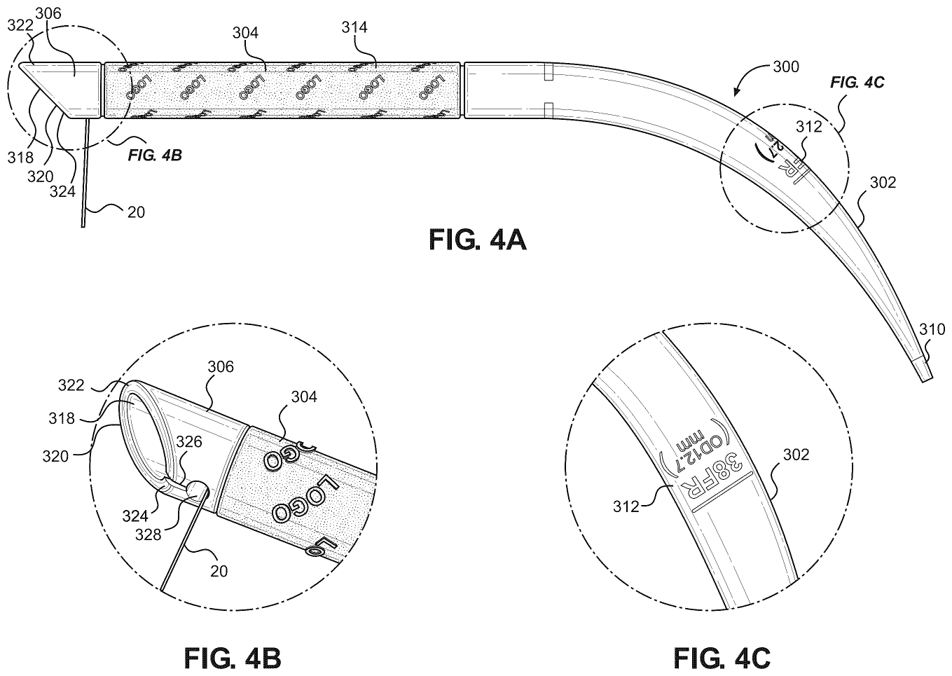

FIGS. 4A-4C illustrate a third exemplary embodiment of a tracheostomy dilator 300. The tracheostomy dilator 300 may include a distal portion 302, a handle portion 304, a proximal portion 306, and a lumen extending therethrough.

The distal portion 302 may including a continuous curve extending from the handle portion 304 to a distal tip 310 having a distal opening of the lumen. The continuous curve may enable the tracheostomy dilator 300 to pass through the trachea during insertion and for the distal tip 310 to clear the posterior tracheal wall. The distal portion 302 may taper along the continuous curve to the distal tip 310 to facilitate insertion. For example, the distal portion 302 may taper through a wall thickness that gradually reduces along the continuous curve to the distal tip 310. The distal portion 302 may have a hydrophilic coating to reduce friction during insertion into the tracheostomy, obviating the need for lubricating jelly. The distal portion 302 may also include indicia 312 of a size of a cross-section and depth of the distal portion 302. For example, as illustrated in FIG. 4C, the indicia 312 may include a diameter (e.g., 38 French) and a line at least partially around the circumference indicating a proper depth for insertion of the distal portion 302 to dilate tracheostomy.

The handle portion 304 may be substantially straight and include an elongated linear shaft integrally formed with the distal portion 302. The handle portion 304 may be proximal of the distal portion 302 and be configured to be grasped by a user. The handle portion 304 may be textured to increase grip and handling of the user. For example, the handle portion 304 may include a textured rubber sleeve 314 over the shaft. The grip on the textured surface may be enhanced by embossing a plurality of raised words and/or logos (e.g., of the manufacturer and/or distributor) along the length of the handle portion 304 and/or sleeve 314. The sleeve 314 may be received in an elongate recess on the outer surface of the shaft to prevent relative slipping.

The proximal portion 306 may be configured to deflect a guidewire 20 at an angle of least 30-45 degrees from a longitudinal axis of the handle portion 304. The proximal portion 306 may be integral to the elongated linear shaft of the handle portion 304 and/or the distal portion 302. The proximal portion 306 may include a proximal opening 318 of the lumen, through which the deflected guidewire 20 may pass out of the tracheostomy dilator 300 when advanced. The proximal opening 318 may include a bevel 320, such that the circumferential wall of the proximal portion 306 extends further proximally at an upper portion 322 than a lower portion 324, effectively shielding the guidewire 20 when grasped from above. The proximal portion 306 may be longitudinally aligned with the longitudinal axis of the handle portion 304, and the bevel 320 may be disposed at about 45 degrees from the longitudinal axis. The lower portion 324 may have a slot 326 and a hole 328, both distal of the proximal opening 318 and in communication with the proximal opening 318 and the lumen. The slot 326 may be narrower than the proximal opening 318 and/or the hole 328. The guidewire 20 may be deflected downward from the proximal opening 318 through the hole 328 and into the slot 326 to reduce or prevent pinching when grasping the handle portion 304 from above.

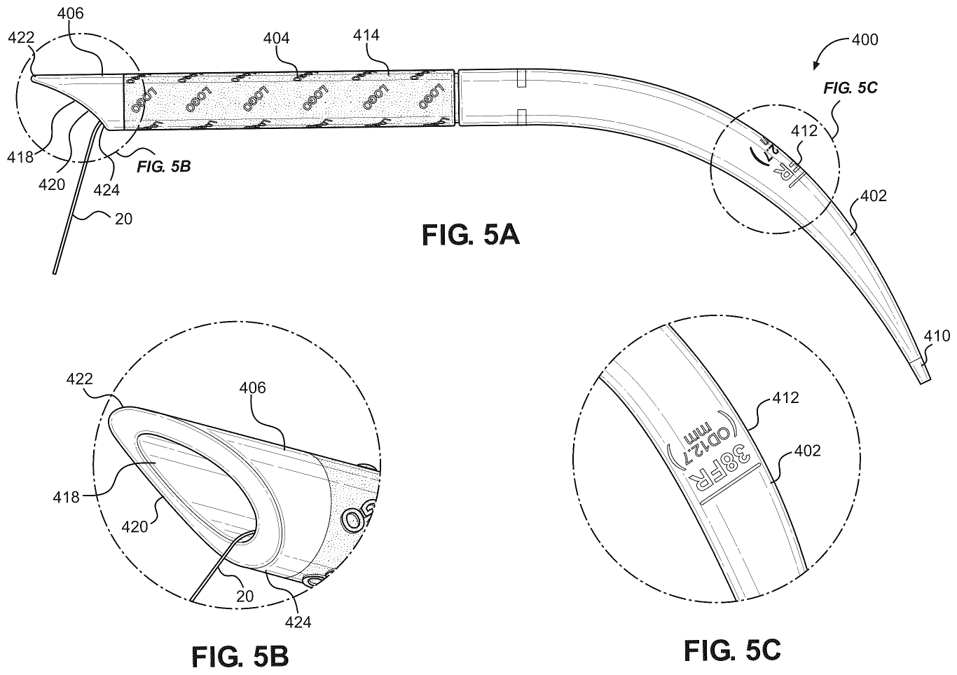

FIGS. 5A-5C illustrate a fourth exemplary embodiment of a tracheostomy dilator 400. The tracheostomy dilator 400 may include a distal portion 402, a handle portion 404, a proximal portion 406, and a lumen extending therethrough.

The distal portion 402 may including a continuous curve extending from the handle portion 404 to a distal tip 410 having a distal opening of the lumen. The continuous curve may enable the tracheostomy dilator 400 to pass through the trachea during insertion and for the distal tip 410 to clear the posterior tracheal wall. The distal portion 402 may taper along the continuous curve to the distal tip 410 to facilitate insertion. For example, the distal portion 402 may taper through a wall thickness that gradually reduces along the continuous curve to the distal tip 410. The distal portion 402 may have a hydrophilic coating to reduce friction during insertion into the tracheostomy, obviating the need for lubricating jelly. The distal portion 402 may also include indicia 412 of a size of a cross-section and depth of the distal portion 402. For example, as illustrated in FIG. 5C, the indicia 412 may include a diameter (e.g., 38 French) and a line at least partially around the circumference indicating a proper depth for insertion of the distal portion 402 to dilate the tracheostomy.

The handle portion 404 may be substantially straight and include an elongated linear shaft integrally formed with the distal portion 402. The handle portion 404 may be proximal of the distal portion 402 and be configured to be grasped by a user. The handle portion 404 may be textured to increase grip and handling of the user. For example, the handle portion 404 may include a textured rubber sleeve 414 over the shaft. The grip on the textured surface may be enhanced by embossing a plurality of raised words and/or logos (e.g., of the manufacturer and/or distributor) along the length of the handle portion 404 and/or sleeve 414. The sleeve 414 may be received in an elongate recess on the outer surface of the shaft to prevent relative slipping.

The proximal portion 406 may be integral to the elongated linear shaft of the handle portion 404 and/or the distal portion 402. The proximal portion 406 may be longitudinally aligned with the longitudinal axis of the handle portion 404. The proximal portion 406 may include a proximal opening 418 of the lumen, through which the guidewire 20 may pass out of the tracheostomy dilator 400 when advanced. The proximal opening 418 may include a bevel 420, such that the circumferential wall of the proximal portion 406 extends further proximally at an upper portion 422 than a lower portion 424, effectively shielding the guidewire 20 when grasped from above. The bevel 420 may be curved.

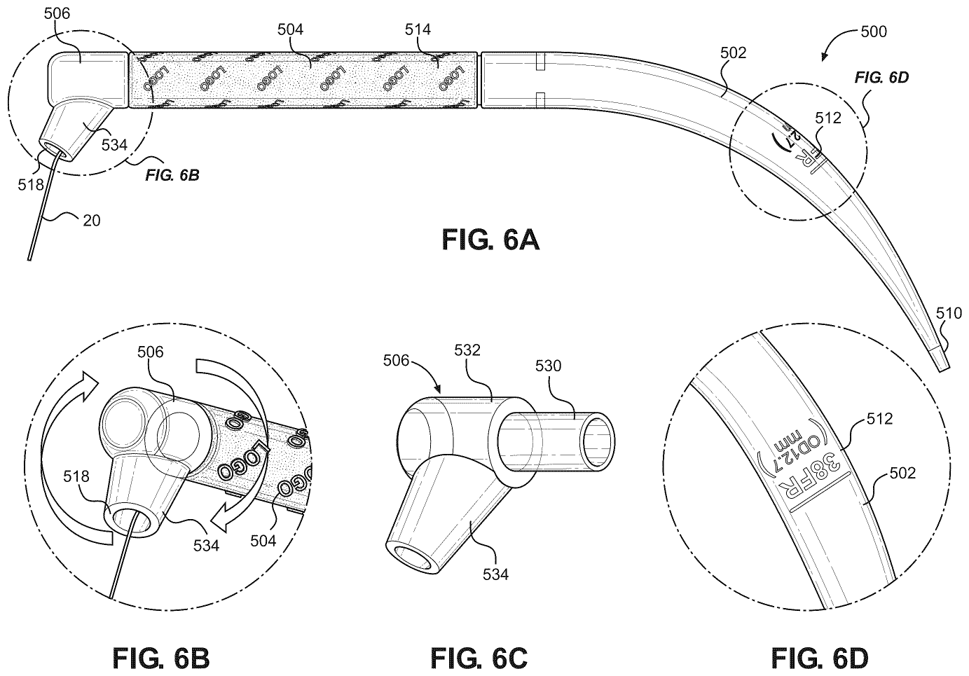

FIGS. 6A-6D illustrate a fifth exemplary embodiment of a tracheostomy dilator 500. The tracheostomy dilator 500 may include a distal portion 502, a handle portion 504, a proximal portion 506, and a lumen extending therethrough.

The distal portion 502 may including a continuous curve extending from the handle portion 504 to a distal tip 510 having a distal opening of the lumen. The continuous curve may enable the tracheostomy dilator 500 to pass through the trachea during insertion and for the distal tip 510 to clear the posterior tracheal wall. The distal portion 502 may taper along the continuous curve to the distal tip 510 to facilitate insertion. For example, the distal portion 502 may taper through a wall thickness that gradually reduces along the continuous curve to the distal tip 510. The distal portion 502 may have a hydrophilic coating to reduce friction during insertion into the tracheostomy, obviating the need for lubricating jelly. The distal portion 502 may also include indicia 512 of a size of a cross-section and a depth of the distal portion 502. For example, as illustrated in FIG. 6D, the indicia 512 may include a diameter (e.g., 38 French) and a line at least partially around the circumference indicating a proper depth for insertion of the distal portion 502 to dilate the tracheostomy.

The handle portion 504 may be substantially straight and include an elongated linear shaft integrally formed with the distal portion 502. The handle portion 504 may be proximal of the distal portion 502 and be configured to be grasped by a user. The handle portion 504 may be textured to increase grip and handling of the user. For example, the handle portion 504 may include a textured rubber sleeve 514 over the shaft. The grip on the textured surface may be enhanced by embossing a plurality of raised words and/or logos (e.g., of the manufacturer and/or distributor) along the length of the handle portion 504 and/or sleeve 514. The sleeve 514 may be received in an elongate recess on the outer surface of the shaft to prevent relative slipping.

The proximal portion 506 may be configured to deflect a guidewire 20 at an angle of least 30-45 degrees from a longitudinal axis of the handle portion 504. The proximal portion 506 may be detachable from the elongated linear shaft of the handle portion 504. For example, the proximal portion 506 may include a distal tubular extension 530 (as shown in FIG. 6C) that is frictionally received in the shaft of the handle portion 504. The distal tubular extension 530 may have a diameter less than a tubular portion 532 that extends uniformly from the handle portion 504. The proximal portion 506 may further include an angled off-axis extension 534 extending from the tubular portion 532 at an angle of at least 30-45 degrees relative to a longitudinal axis of the handle portion 504. The angled extension 534 may include a proximal opening 518 of the lumen, through which the deflected guidewire 20 passes out of the tracheostomy dilator 500 when advanced. As illustrated in FIG. 6B, the proximal portion 506 may be rotated about the longitudinal axis of the proximal portion 506 to position the angled extension 534 and proximal opening 518 in any radial direction. The rotation of the proximal portion 506 allows the reorientation of the guidewire 20, for example, depending on the desired grip of the user and/or whether the user is right-handed or left-handed. Thus, the proximal portion 506 may reduce or prevent pinching of the guidewire 20 when grasping the handle portion 504.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A tracheostomy dilator comprising:
a distal portion having a curve extending to a distal opening and configured to dilate a tracheostomy tracheostoma;
a handle portion proximal of the distal portion, defining a longitudinal axis, and configured to be grasped by a user;
a lumen configured to receive a guidewire; and
a proximal portion proximal of the handle portion and having a proximal opening in communication with the lumen, wherein the proximal portion is configured to allow deflection of the guidewire when the guidewire exits the dilator through the proximal opening at a proximal end of the handle portion, away from the longitudinal axis of the handle portion,
wherein the proximal portion is beveled at a face of the proximal opening such that a circumferential wall of the proximal portion defining the proximal opening extends further proximally at an upper portion than a lower portion.

2. The tracheostomy dilator of claim 1, wherein the guidewire can be deflected at an angle of at least 30 degrees from a longitudinal axis of the handle portion.

3. The tracheostomy dilator of claim 2, wherein the angle is at least 45 degrees from the longitudinal axis of the handle portion.

4. The tracheostomy dilator of claim 1, wherein the handle portion is straight.

5. The tracheostomy dilator of claim 1, wherein the handle portion is textured.

6. The tracheostomy dilator of claim 5, wherein the handle portion includes a textured sleeve.

7. The tracheostomy dilator of claim 1, wherein the distal portion has a wall thickness that decreases along a length of the distal portion.

8. The tracheostomy dilator of claim 1, wherein a bevel at the face of the proximal opening is curved.

9. The tracheostomy dilator of claim 1, further comprising an indicator of a size of the tracheostomy dilator on the distal portion.

10. The tracheostomy dilator of claim 1, wherein the proximal portion is longitudinally aligned with the longitudinal axis of the handle portion.

11. The tracheostomy dilator of claim 1, wherein the proximal portion is rotatable around the longitudinal axis relative to the handle portion.

12. The tracheostomy dilator of claim 1, wherein the proximal portion is detachable from the handle portion.

13. The tracheostomy dilator of claim 1, wherein the distal portion is curved.

14. The tracheostomy dilator of claim 1, wherein the guidewire can be deflected at an angle of at least 45 degrees from a longitudinal axis of the handle portion, and wherein the distal portion is curved, and wherein the distal portion has a wall thickness that decreases along a length of the distal portion.

15. The tracheostomy dilator of claim 14, wherein the handle portion is textured.

16. The tracheostomy dilator of claim 15, wherein the handle portion is straight.
